(19) United States
(12) Patent Application Publication
(10) Pub. No.: US 2008/0269514 A1
(43) Pub. Date: Oct. 30, 2008

(54) Iron or Cobalt-Catalyzed Carbon-Carbon Coupling Reaction of Aryls, Alkenes and Alkines With Copper Reagents

(75) Inventors: Paul Knochel, Munchen (DE); Ioannis Sapountzis, Munich (DE); Tobias Korn, Neuried (DE); Wenwei Lin, Munich (DE); Christiane Charlotte Kofink, Munich (DE)

Correspondence Address: LANXESS CORPORATION, 111 RIDC PARK WEST DRIVE, PITTSBURGH, PA, 15275-1112, US

(73) Assignee: SALTIGO GMBH, Langenfield (DE)

(21) Appl. No.: 11/576,930

(22) PCT Filed: Oct. 11, 2005
(86) PCT No.: PCT/EP2005/010936

§ 371 Date: Feb. 14, 2008

(30) Foreign Application Priority Data
Oct. 11, 2004 (DE) 10 2004 049 508.4

(51) Int. Cl. C07C 69/76; C07C 59/48; C07C 41/05; C07C 63/74

(52) U.S. Cl. 560/102; 562/469; 568/658; 562/495

(57) ABSTRACT

The present invention relates to a process for the formation of carbon-carbon bonds starting from a copper compound of an aryl, heteroaryl, alkene or alkine and an aryl, heteroaryl, alkene or alkine compound having a suitable leaving group. The copper compounds can be prepared inter alia by means of transmetalliziation from a Grignard or lithium compound. Cross-coupling of these compounds with e.g. a halogen-substituted aryl compound is carried out by means of an iron or cobalt catalyst using suitable solvents and suitable additives.

### DESCRIPTION OF THE INVENTION

The present invention relates to a process for the formation of carbon-carbon bonds starting from a copper compound of an aryl, heteroaryl, alkene or alkine and an aryl, heteroaryl, alkene or alkine compound having a suitable leaving group. The cross-coupling of these compounds using e.g. a halogen-substituted aryl compound is carried out by means of an iron or cobalt catalyst using suitable solvents.

### BACKGROUND OF THE INVENTION

Transition metal catalyzed cross-couplings are very powerful reactions for the formation of C—C bonds, in particular between Csp² centers at which typical S_{N}² substitutions are not possible.^{[1]} Aryl-aryl cross-coupling is one of the most important methods for the formation of carbon-carbon bonds. Many aromatics obtained in this manner and in particular the heteroaromatics are of high interest both for the agricultural and the pharmaceutical industries as well as for material science. For this purpose, most frequently reliable palladium(0) catalysts are used,^{[1,2]} mostly in the presence of an appropriate ligand, such as for example sterically hindered phosphines.^{[3]} Also nickel (0) complexes find practical use but seem to have a less wider utility.^{[4]} Since palladium is expensive and nickel is toxic there is a need for cheap an non-toxic catalysts. Iron and cobalt salts are such cheap and non-toxic alternatives.

Following the work of Kochi,^{[5]} intensive research has been directed recently to the performance of iron catalyzed cross-couplings in cross-coupling reactions.^{[6]} Although very efficient cross-couplings could be achieved between a number of alkyl magnesium reagents and aryl halogenides or aryl sulfonates, the catalyzed cross-coupling between two aryl residues remained problematic due to the substantial homocoupling reaction of the aryl magnesium species, and up to now no synthetic solution for this problem has been found.^{[6,7]} However, in this case also a dehalogenation of the aryl halogenides takes place.

For this reason, the inventors have transmetallized the aryl magnesium species onto the corresponding organozinc compound which has a reduced tendency for the formation of unstable At complexes.^{[8]} However, a catalyzed cross-coupling of aryl zinc reagents using aryl halogenides could not be observed under various reaction conditions. Therefore, it is the object of the present invention to provide a simple process for the directed formation of carbon-carbon bonds between arylene, alkenes and alkines in high yields and at low costs.

According to the invention, this object has been achieved by claim 1. Preferred embodiments are described in the dependent claims.

### SUMMARY OF THE INVENTION

The inventors drew their attention to other classes of organo-metallic compounds and found that organocopper compounds^{[9]} of type 1 react with different functionalized aryl halogenides (3) in the presence of catalytic amounts of Fe or Co in a polar solvent or solvent mixture and result in polyfunctional biaryls of type 4 (Table 1 and 2).

R¹—Ar¹—Ar²—R² (4)

Organocopper compounds of type 1 can be prepared by the reaction of functionalized aryl magnesium halogenides (2)^{[10]} with CuCN.2LiCl^{[11]}. Here and in the following, the term aryl is intended to mean aryl, heteroaryl, alkene, or alkine. These compounds can be mono- or polysubstituted. An essential feature of the invention is the presence of an aryl, alkene or alkine compound whose characteristic aryl, alkene or alkine features are utilized by the reaction mechanism.

A first aspect of the present invention relates to a process of preparation of a compound of the general formula 4

R¹—Ar¹—Ar²—R² (4)

by reacting a compound of the general formula (I)

R¹—Ar¹—CuZMgY (1)

or a compound of the general formula (5)

R¹—Ar¹—CuZLi (5)

with a compound of the general formula (3)

R²—Ar²—X (3)

under the action of a Co or Fe catalyst in a solvent wherein X may be a leaving group useful for nucleophilic substitution;
Y may be Cl, Br, I;
Z may be CN, Cl, Br, I, SCN, NR¹R², SR¹, PR¹R², alkyl, alkinyl;
R¹ and R² independently of each other may be one or more substituents selected from H; substituted or unsubstituted aryl or heteroaryl containing one or more heteroatoms; straight-chain, branched or cyclic substituted or unsubstituted alkyl, alkenyl, alkinyl; or derivatives thereof;
Ar and Ar² independently of each other may be an aryl, condensed aryl, heteroaryl or condensed heteroaryl containing one or more heteroatoms; an alkenyl or alkinyl; or derivatives thereof.

The leaving group X represents a leaving group which is conventionally used in a nucleophilic substitution. The group referred to as Ar can also be substituted with several substituents fulfilling the definition of R, if practicable.

According to one embodiment of the invention the reaction is performed at a temperature between 0° C. and 150° C., preferably between 10° C. and 120° C., even more preferably between 20° C. and 100° C., most preferably between 25° C. and 80° C.

According to another embodiment the catalyst comprises a Fe(III) complex, a Fe(III) salt, a Fe(II) complex, a Fe(II) salt, or a reduced form of a Fe salt or complex, preferably Fe(acac)₃. As the iron compounds, any iron(II) or iron(III) salt or complex can be employed such as e.g. FeCl₂, FeCl₃, FeBr₂, FeBr₃, Fe(OAc)₂, Fe(OAc)₃, etc., as well as other iron complexes containing iron in other oxidation stages, and also reduced iron complexes in which the iron has a negative oxidation stage.

According to another embodiment the catalyst comprises a Co(II) or Co(III) catalyst.

According to another embodiment the catalyst is selected from the group comprising CoCl₂, CoBr₂, Co(OAc)₂, Co(Bzac)₂, CoBr₂dppe, Co(acac)₂ and Co(acac)₃, and preferably Co(acac)₂ is used.

As the cobalt compounds, cobalt salts or complexes having any oxidation stage of cobalt can be used, also cobalt complexes in which the cobalt has a negative oxidation stage.

According to another embodiment ethene and/or one or more ethene derivative(s), preferably electron-deficient ethene derivatives, particularly preferred maleic anhydride, tetracyano-ethylene, styrene or a styrene derivative, even more preferably an electron-deficient styrene derivative, and most preferably 4-fluorostyrene, are additionally added during the catalytic reaction.

According to another embodiment the ethene or ethene derivative is added in an amount of 0-50 mole %, preferably 1-30 mole %, particularly preferred 5-25 mole %, most preferably 10-20 mole %, based on the molar amount of compound (3).

According to another embodiment one or more salts, preferably tetrabutylammonium chloride, tetrabutylammonium bromide, potassium iodide, lithium iodide and/or most preferably tetrabutylammonium iodide are additionally added during the catalytic reaction.

According to another embodiment X can preferably be F, Cl, Br, I, OTf, OTs, N₂⁺, more preferably Cl or Br, even more preferably I.

According to another embodiment X can preferably be F, Cl, Br, I, OTf, OTs, N₂⁺, more preferably F, Cl, I or OTs, even more preferably Br.

According to another embodiment, a polar solvent or solvent mixture, preferably an etheric solvent or solvent mixture and most preferably a solvent or solvent mixture selected from the group comprising THF, DME, NMP, DMPU and DMAC is used as the solvent.

According to another embodiment the compound (1) or (5) is added in a molar ratio of 0.9-5, preferably in a molar ratio of 1-3, even more preferably in a molar ratio of 1.2-2.5 based on the molar amount of compound (3).

According to another embodiment Z preferably is CN.

According to another embodiment R¹ and R² independently of each other can be one or more substituted or unsubstituted C₄-C₂₄ aryls or C₃-C₂₄ heteroaryls containing one or more heteroatoms such as B, O, N, S, Se, P; straight-chain or branched, substituted or unsubstituted C₁-C₂₀ alkyls, C₁-C₂₀ alkenyls, C₁-C₂₀ alkinyls; or substituted or unsubstituted C₃-C₂₀ cycloalkyls; or derivatives thereof.

This novel procedure enables an economical approach (about three times cheaper as compared to Pd catalyzed reactions) for carrying out aryl-aryl cross-couplings.

### DETAILED DESCRIPTION OF THE INVENTION

In the following, the present invention will be described in more detail referring to the Figures.

If not specified otherwise, the technical and scientific terms used herein are intended to have the same meaning as understood by those skilled in the field to which this invention refers.

### Iron Catalyzed Reaction Scheme

Organocopper compounds^{[9]} of type 1 which are prepared by the reaction of functionalized aryl magnesium chlorides (2)^{[10]} using CuCN.2LiCl^{[11]} can be reacted with different functionalized aryl halogenides (3) in the presence of catalytic amounts of Fe(acac)₃ (10 mol %) in a mixture of DME:THF (3:2) and lead to the polyfunctional biphenyls of type 4 (Table 1 and 2):

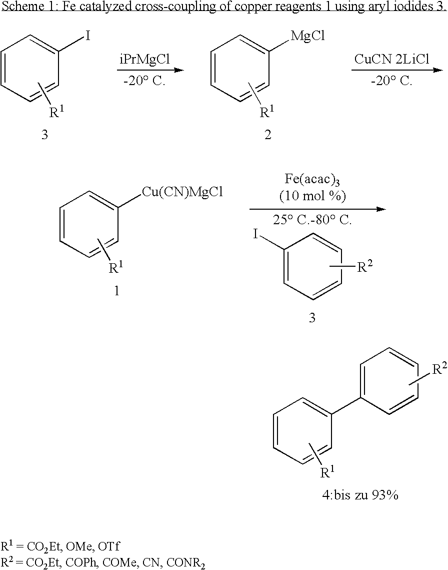

The reaction proceeds between 25° C. and 80° C. If organocopper reagents (1) are used the extent of homo coupling is markedly reduced and the cross-coupling takes place straightforwardly. The type of the leaving group of the aromatic electrophilic 3 is also important (Table 1). Therefore, the reaction of PhCu(CN)MgCl (1a) with 2-iodobenzophenone (3a) is complete within 30 min at 25° C. Without Fe(acac)₃ less than 5% of the biphenyl 4a is observed after 30 min, and after a reaction time of 48 h only approx. 54% conversion can be observed (entry 1 in Table 1). The corresponding bromide (2-bromobenzophenone; entry 2) also reacts quickly but results in a conversion of only 86% after 30 min. A reaction time of 18 h achieves only a little improvement of the conversion (93%). Similarly, also the 2-chlorobenzophenone does not result in complete conversion (75% after 30 min, but only 77% after 18 h). A significantly slower reaction can be observed if a triflate substituent (X═OTf) is used. The fact that 2-chlorobenzophenone is able to react suggests that the reaction mechanism does not involve a halogen-copper exchange reaction. No reaction could be observed for 2-chlorobenzophenone in the absence of the iron catalyst.

Interestingly, for the reaction of aryl copper compounds with aryl halogenides the inventors have observed a reversed reactivity as compared to Fe(III) catalyzed reactions with alkyl magnesium compounds for which aryl iodides are less suitable as substrates than aryl bromides or chlorides.^{[7]} Subsequently, the inventors have investigated the range of use of the reaction and have found a remarkable chemoselectivity and compatibility with functional groups. The reaction of PhCu(CN)MgCl (1a) with 2-iodobenzophenone (3a) is particularly fast (30 min) and the corresponding ketone 4a can be isolated in a yield of 93% (entry 1 in Table 2). 4-Iodobenzophenone (3b) reacts within 4 h at 25° C. and yields the desired ketone 4b in an amount of 80% (entry 2). Conspicuously, a methyl ketone such as 2-iodophenylmethyl ketone (3c) undergoes the iron catalyzed cross-coupling without any competing deprotonation of the methyl ketone or addition to the carbonyl function. Functionalized aryl magnesium reagents having an ester group in ortho or para position, such as 1b, 1c or 1d, also undergo the cross-coupling within a few hours and result in the expected products 4d-f in a yield of 68-86% (entries 4-6). The sterically hindered functionalized 2,2′-substituted biphenyl 4d thus is prepared in a yield of 75% (entry 4). Also Grignard reagents carrying a donor group, such as 1e, react well and yield the ketone 4g in an amount of 76% (entry 7). Various para-substituted aryl iodides carrying an electron-attracting group such as a cyanide (3e), an amide (3f) or a ketone (3b) react in a mild cross-coupling reaction and yield products 4h-j in amounts of 50-70% (entries 8-10). Surprisingly, the copper-reagent if having a triflate group reacts with ethyl-2-iodobenzoate 3g yielding biphenyl 4k in an amount of 62% (entry 11). The inventors have found that the presence of an electron-attracting substituent on the aryl iodide accelerates the cross-coupling and that donor substituents substantially slow down the reaction. Therefore, 4-iodoanisole only results in a low conversion at 80° C. after 12 h. The inventors have also compared the reaction rate of the cross-coupling for 2-, 3- and 4-substituted ethyl iodobenzoate (3g-i) with PhCu(CN)MgCl in the presence of Fe(acac)₃ (10 mole %) (Scheme 2). Interestingly, the ortho- and para-substituted iodobenzoates 3g and 31 react much faster as opposed to the meta-substituted ester 3h showing a reaction time which is five times as long until complete conversion. This could indicate that the slowest step in cross-coupling is the nucleophilic attack of the catalytically active species at the benzoate 3g-i. The inventors of the present invention have performed the Pd and Ni catalyzed cross-coupling also using the substituted iodoesters 3g-i and have found that in the case of Pd catalysis the para-iodoester 3i reacts significantly faster than 3g and 3h whereas under conditions of Ni catalysis the meta-iodoester 3h reacts fastest. These results suggest that the mechanisms of the Ni, Pd and Fe catalyzed cross-coupling reactions are very different since they are affected in a different manner by the electronic and steric effects of the substituents.

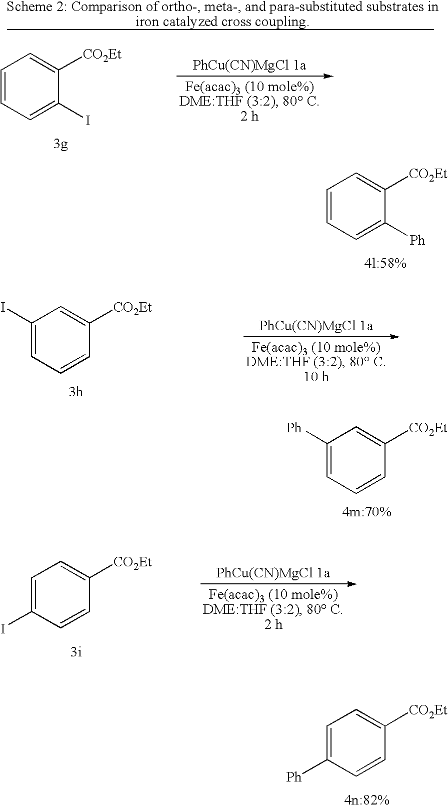

Heterocyclic compounds have also been found to be suitable for this cross-coupling reaction. Accordingly, the copper reagents 1g and 1h react under standard conditions with iodides 3f and 3e and result in the functionalized indole 4o and the pyridine 4p in yields of 85% and 57%, respectively (Scheme 3).

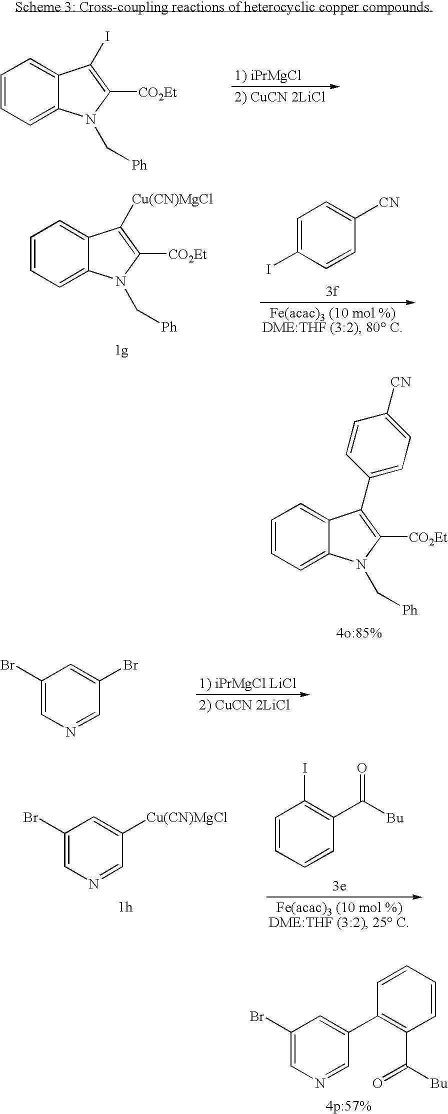

The iron catalyzed cross-coupling of heteroaryl and aryl copper compounds prepared from the corresponding organomagnesium compounds proceeds conveniently if functionalized aryl iodides are used. In this respect, the electron-deficient electrophilic iodides undergo the cross-coupling reaction much more easily.

### Cobalt Catalyzed Reaction Scheme

As demonstrated by the following examples, a Co salt can also be used as the catalyst.

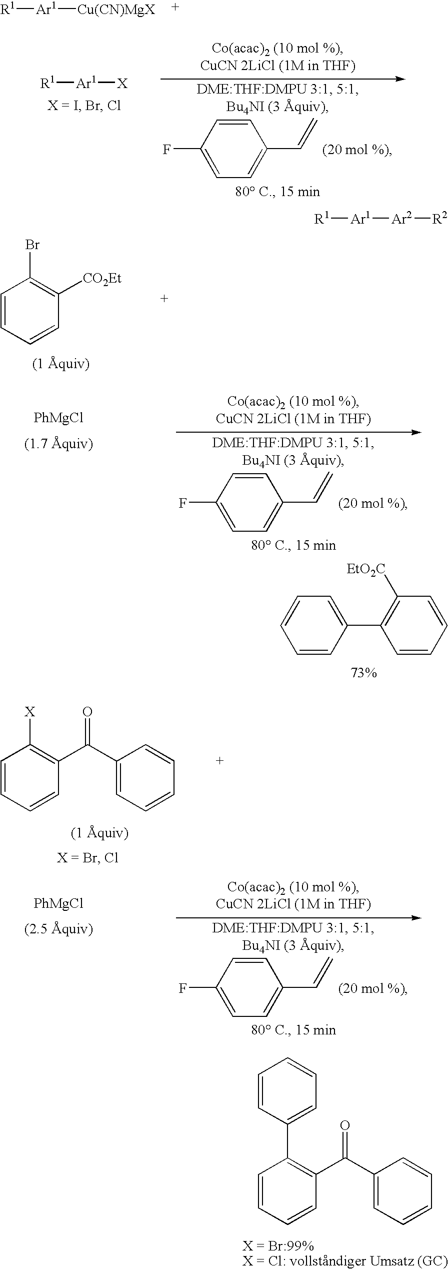

A key step in the aryl-aryl coupling between functionalized aryl or heteroaryl magnesium compounds with functionalized aryl or heteroaryl halogenides is the transmetallization of the aryl magnesium compound (R¹—ArMgCl) onto copper to obtain copper organyls of the type R¹—ArCu(CN)MgX in this manner. In addition to the copper salt CuCN also the salts CuCl, CuBr, Cul, CuSCN etc. can be used as a copper source for transmetallization.

The reaction is carried out in a solvent mixture of THF, DME and a polar co-solvent. For such purpose, NMP, DMPU but also DMAC are employed.

Further important components of the reaction are the addition of a salt such as tetrabutylammonium iodide and an electron-deficient styrene derivative such as 4-fluorostyrene. In addition to tetrabutylammonium iodide also tetrabutylammonium bromide, tetrabutylammonium chloride, potassium iodide or lithium iodide can be used.

The catalyst is represented by a cobalt salt. In this respect, Co(acac)₂ has been found to be the most suitable. However, the reaction is also catalyzed by CoCl₂, CoBr₂, Co(OAc)₂, Co(Bzac)₂, CoBr₂dppe or Co(acac)₃.

The coupling partners for the functionalized organocopper compounds are functionalized aryl halogenides. For this purpose, iodides, bromides and partially chlorides can be employed. Most important in this respect are the bromides and chlorides since they are generally cheaper and thus more interesting for industrial applications than iodides.

Using the process for cross-coupling reactions between aryl bromides and chlorides and aryl copper and aryl magnesium compounds, respectively, which has already been described in this application also aryl fluorides and tosylates (OTs) can be reacted. However, 3 equivalents of the aryl copper compound are required for these reactions.

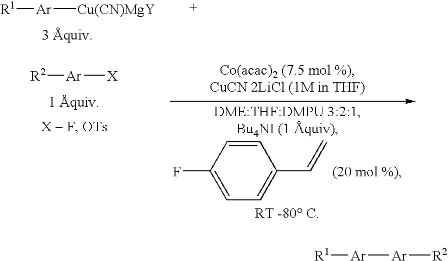

If 2,3,4,5,6-pentafluorobenzophenone 5h is used two cross-coupling reactions can be performed simultaneously. Accordingly, 6 equivalents of copper reagent are required as can be seen from Scheme 4.

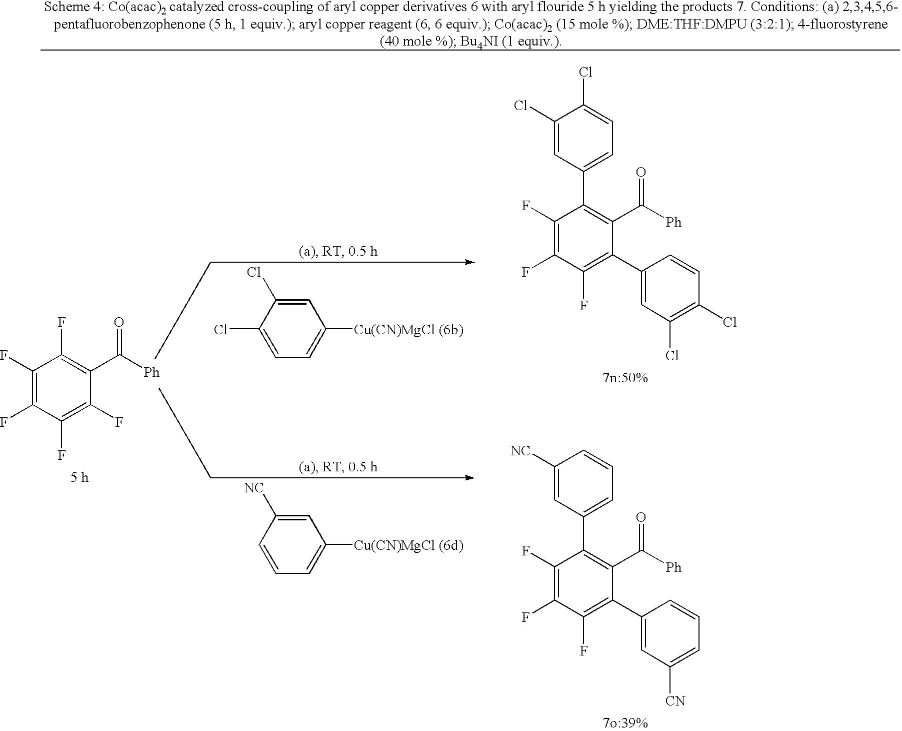

The use of the polar co-solvents, the salts and the styrene derivative as well as the cobalt salt is essential for the coupling reaction to be carried out completely and quickly with bromides and chlorides. Without these additions no complete reaction occurs or it will take days instead of a few minutes or hours.

### EXAMPLE 1

### Synthesis of 4′-Cyanobiphenyl-4-carboxylic ethyl ester (4h)

Ethyl-4-iodobenzoate (855 mg, 3.10 mmoles) is charged into a 25 mL Schlenk tube with magnetic stirring bar and septum, DME (5 mL) is added, and the solution is cooled to −20° C. Afterwards, iPrMgCl (3.3 mL, 3.0 mmoles, 0.90 M in THF) is added. The reaction mixture is stirred for 15 min at this temperature, and then a CuCN.2LiCl solution (2.8 mL, 2.8 mmoles, 1 M in THF) is added. The reaction mixture is stirred for another 10 min. A solution of 4-iodobenzonitrile (229 mg, 1.00 mmoles) and Fe(acac)₃ (35 mg, 0.10 mmoles) dissolved in DME (3 mL) is added at once and then the reaction mixture is heated for 3 h to 80° C. The reaction is terminated by the addition of sat. NH₄Cl(aq.) and extracted with CH₂Cl₂ (3×40 mL). The combined organic phases are washed with sat. NH₄Cl(aq.)/NH₃ (9:1) (50 mL) and sat. NaCl(aq.) (50 mL), dried over Na₂SO₄, filtered and the solvent is distilled-off under reduced pressure. Purification by column chromatography (pentane/diethylether=9:1) yielded 4h as a colourless solid (181 mg, 72%).

### EXAMPLE 2

### Reaction Rates Achieved with Different Leaving Groups X

Following the experimental protocol of Example 1 reactions were performed with benzophenones carrying different substituents. The appropriate substituents at the 2-position are listed in Table 1 together with the conversions achieved therewith.

TABLE 1
Reaction rate of 2-substituted benzophenones with PhCu(CN)MgCl
in the presence of Fe(acac)₃.

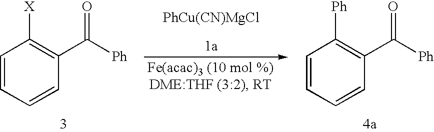

| Entry | X | Conversion (%)^{[a]} |
|---|---|---|
| 1 | I | 100, (<5)^{[b]}, (55)^{[c]} |
| 2 | Br | 86, (93)^{[d]} |
| 3 | Cl | 75, (77)^{[d]} |
| 4 | OTf | 35, (100)^{[e]} |
| 5 | OTs | 0 |

^{[a]}Conversion after 30 min reaction time as determined by GC analysis of reaction samples;
^{[b]}conversion in the absence of Fe(acac)₃ after 30 min;
^{[c]}conversion after 48 h in the absence of Fe(acac)₃;
^{[d]}conversion after 18 h of reaction time
^{[e]}conversion after 2 h of reaction time in THF.

### EXAMPLE 3

### Reactions under Fe Catalysis Conditions

Following the experimental protocol in Example 1 all compounds represented in Table 2 were synthesized. The appropriate educts 1 and 2 as represented in the Table were reacted to the corresponding products 4. The yield of the reaction is listed in the last column of the Table.

TABLE 2
Type 4 products obtained by Fe catalyzed cross-coupling of functionalized aryl
copper compounds (1) with aryl iodides.

| | aryl cuprate | aryl iodide | product of type | Yield |
|---|---|---|---|---|
| Entry | 1^{[a]} | 3 | 4 | (%)^{[b]} |
| 1 | PhCu1a | 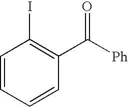 3a | 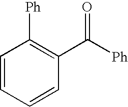 4a | 93 |
| 2 | PhCu1a | 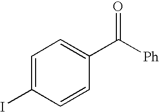 3b | 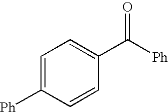 4b | 80 |
| 3 | PhCu1a | 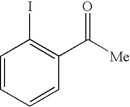 3c | 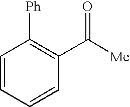 4c | 86 |
| 4 | 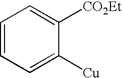 1b | 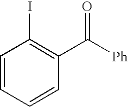 3a | 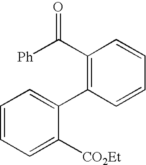 4d | 75 |
| 5 | 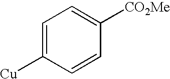 1c | 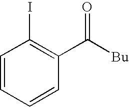 3d | 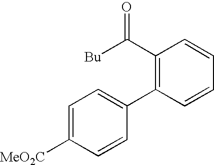 4e | 68 |
| 6 | 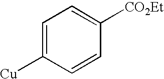 1d | 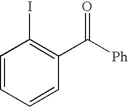 3a | 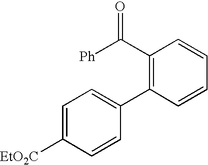 4f | 86 |
| 7 | 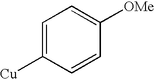 1c | 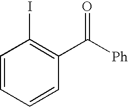 3a | 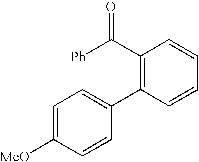 4g | 76 |
| 8 | 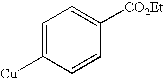 1d | 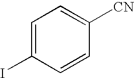 3e | 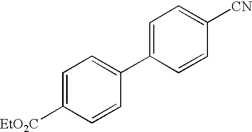 4h | 72 |
| 9 | 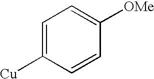 1e | 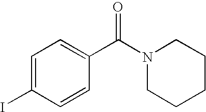 3f | 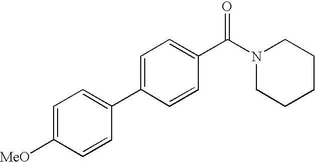 4i | 58 |
| 10 | 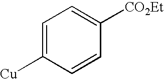 1d | 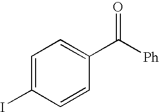 3b | 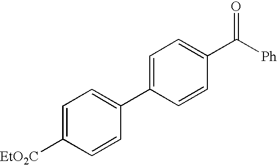 4j | 50 |
| 11 | 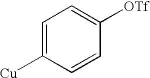 1f | 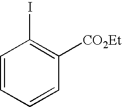 3g | 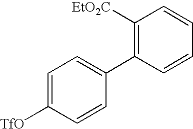 4k | 62 |

^{[a]}The copper reagent is better represented by ArCu(CN)MgCl.
^{[b]}Yield of analytically pure product.

### EXAMPLE 4

### Synthesis of 1,1′-Biphenyl-2-carboxylic ethyl ester

DME (5.0 mL) is charged in a 25 mL Schlenk tube with magnetic stirring bar and septum, PhMgCl (1.42 mL, 1.70 mmoles, 1.2 M in THF) and a CuCN.2LiCl solution (1.90 mL, 1.90 mmoles, 1 M in THF) are added, and the solution is stirred for approx. 10 min at room temperature. Thereafter, DMPU (2.0 mL), tetrabutylammonium iodide (1.11 g, 3.00 mmoles), 4-fluorostyrene (25 mg, 0.21 mmoles), 2-bromobenzoic acid ethyl ester (229 mg, 1.00 mmoles) and a solution of Co(acac)₂ (25.8 mg, 0.10 mmoles) in DME (1.0 mL) are added. The resulting suspension is heated for 15 min to 80° C. The reaction mixture is extracted with sat. NH₄Cl(aq.)/NH₃ (4:1) (2×50 mL), the combined organic phases are extracted with diethylether (3×50 mL), and the combined organic phases are washed sat. NaCl(aq.) (50 mL). Afterwards drying over Na₂SO₄ an filtration are carried out, and the solvent is distilled-off under reduced pressure. Purification by column chromatography (pentane/diethylether=19:1) yielded the product as a colourless solution (165 mg, 73%).

### EXAMPLE 5

### Synthesis of 2′-(4-Methoxybenzoyl)-biphenyl-4-carbonitrile (7g)

Into a 25 mL Schlenk flask equipped with stirring bar and septum iPrMgCl LiCl (2.63 mL, 3.15 mmoles, 1.2 M in THF) was charged. The solution was cooled to −20° C. and 4-bromobenzonitrile (544 mg, 2.99 mmoles) was added. Afterwards, the reaction mixture was warmed to 0° C. and stirred for 2 h at this temperature. Then, a CuCN.2LiCl solution (3.2 mL, 3.2 mmoles, 1 M in THF) was added. After 10 min DME (6.0 mL), DMPU (2.0 mL), Bu₄NI (370 mg, 1.00 mmoles), 4-fluorostyrene (25 mg, 0.20 mmoles), Co(acac)₂ (19.3 mg, 0.075 mmoles) and (2-fluorophenyl)(4-methoxyphenyl)methanone (230 mg, 1.00 mmoles) were added. The reaction mixture was stirred for 0.25 h at room temperature and subsequently quenched with a sat. NH₄Cl(aq.)/NH₃ mixture (9:1) (50 mL). The organic phase was washed once again with the sat. NH₄Cl(aq.)/NH₃ mixture (9:1) (50 mL) and the combined aqueous phases were extracted with EtOAc (3×40 mL). The combined organic phases were washed with sat. NaCl solution (50 mL), dried over MgSO₄, filtered from the drying agent and the solvent was concentrated in vacuum. The purification by column chromatography on silica (pentane/diethylether=4:1) yielded 7g as a colourless solid (271 mg, 0.87 mmoles, 87%, m. p.: 120.8-122.9° C.).

### EXAMPLE 6

### Reaction Under Co-Catalysis Conditions

Following the experimental protocol in Example 5 above all products 7 listed in Table 3 can be prepared. For this purpose, the appropriate educts 5 and 6 as well as the conditions according to the Table must be substituted in the protocol of Example 5. The yield is given in the right column of Table 3.

TABLE 3
Co(acac)₂ catalyzed cross-coupling of aryl copper derivatives 6 with aryl fluorides 5
to obtain the products 7.

| | | aryl copper | | conditions | Yield |
|---|---|---|---|---|---|
| Entry | aryl fluorides 5 | reagent 6^{[a]} | Product 7 | [° C., h] | [%]^{[b]} |
| 1 | 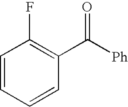 5a | 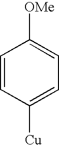 6a | 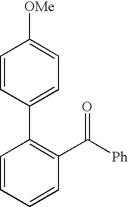 7a | 25, 2 | 98 |
| 2 | 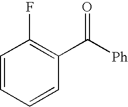 5a | 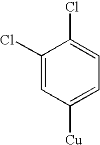 6b | 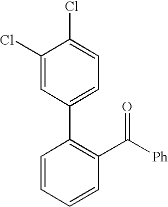 7b | 25, 0.5 | 87 |
| 3 | 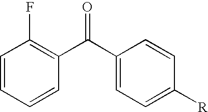 5b: R = OPiv | 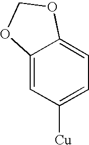 6c | 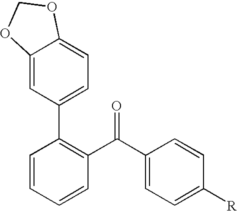 7c: R = OPiv | 25, 16 | 72 |
| 4 | 5c: R = OMe | 6c | 7d: R = OMe | 25, 3 | 98 |
| 5 | 5d: R = NMe₂ | 6c | 7e: R = NMe₂ | 25, 3 | 95 |
| 6 | 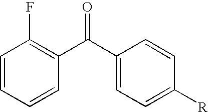 5b: R = OPiv | 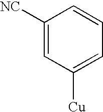 6d | 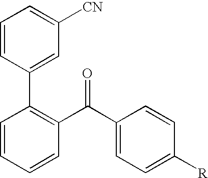 7f: R = OPiv | 25, 0.25 | 52 |
| 7 | 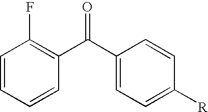 5c: R = OMe | 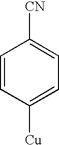 6e | 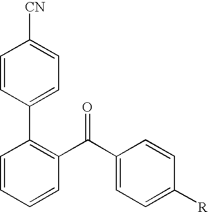 7g: R = OMe | 25, 0.25 | 87 |
| 8 | 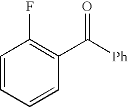 5a | 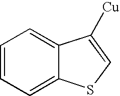 6f | 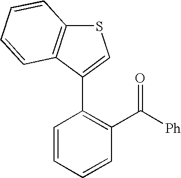 7h | 80, 1 | 94 |
| 9 | 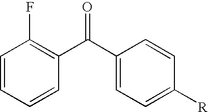 5d: R = NMe₂ | 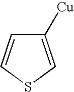 6g | 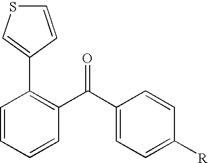 7i: R = NMe₂ | 80, 1 | 42 |
| 10 | 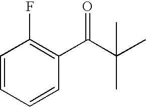 5e | 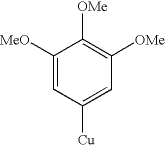 6h | 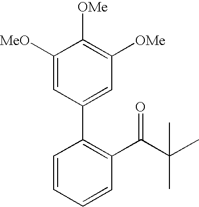 7j | 80, 1 | 71 |
| 11 | 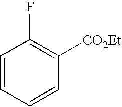 5f | 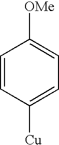 6a | 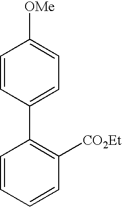 7k | 80, 0.25 | 51 |
| 12 | 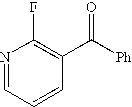 5g | 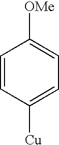 6a | 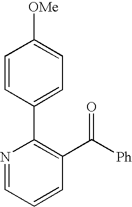 7l | 80, 1 | 25 |
| 13 | 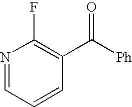 5g | 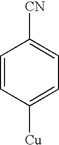 6e | 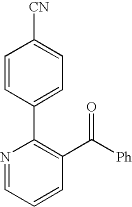 7m | 80, 3 h | 15 |

^{[a]}The copper reagent is better represented by ArCu(CN)MgCl.
^{[b]}Yield of analytically pure product.

### EXAMPLE 7

### Synthesis of (E)-1-(Hex-1-enyl)-4-methoxybenzene

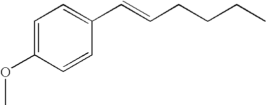

4-Methoxyphenyl Grignard (3.75 mL, 3.00 mmoles, 0.8 M in THF) in DME (5 mL) is charged into a 25 mL Schlenk tube equipped with magnetic stirring bar and septum, the solution is cooled to −20° C., and then a CuCN.2LiCl solution (2.8 mL, 2.8 mmoles, 1 M in THF) is added. The reaction mixture is stirred for 10 min. Subsequently, a solution of 4-iodohexene (210 mg, 1.00 mmoles) and Fe(acac)₃ (35 mg, 0.10 mmoles) dissolved in DME (3 mL) is added at once and then the reaction mixture is heated for 3 h to 80° C. The reaction is terminated by the addition of sat. NH₄Cl(aq.) and extracted with CH₂Cl₂ (3×40 mL). The combined organic phases are washed with sat. NH₄Cl(aq.)/NH₃ (9:1) (50 mL) and sat. NaCl(aq.) (50 mL), dried over Na₂SO₄, filtered and the solvent is distilled-off under reduced pressure. Purification by column chromatography (pentane/diethylether=19:1) yielded (E)-1-(Hex-1-enyl)-4-methoxybenzene as a colourless oil (118 mg, 62%).

¹H-NMR (300 MHz, CDCl₃, 25° C.): δ=7.19 (d, J=8.7 Hz, 2H), 6.75 (d, J=8.7 Hz, 2H), 6.24 (d, J=15.4 Hz, 1H), 6.04-5.94 (m, 1H), 3.70 (s, 3H), 2.00 (q, J=6.8 Hz, 2H), 1.41-1.24 (m, 5H), 0.84 (t, J=7.7 Hz, 3H).

¹³C-NMR (75 MHz, CDCl₃, 25° C.): δ=158.58, 130.82, 129.01, 126.93, 113.88, 55.23, 32.67, 31.66, 22.24, 13.93.

MS (70 eV, EI): m/z (%):190 (37) [M], 147 (100), 134 (10), 115 (11), 103 (5), 91 (14), 77 (4), 65(2).

HRMS for C₁₃H₁₈O (190,1358): found: 190.1337.

### EXAMPLE 8

### Synthesis of (Z)-Ethyl-3-(4-methoxyphenyl)but-2-enoate

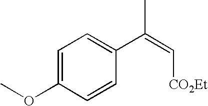

4-Methoxyphenyl Grignard (3.75 mL, 3.00 mmoles, 0.8 M in THF) in DME (5 mL) is charged into a 25 mL Schlenk tube equipped with magnetic stirring bar and septum, the solution is cooled to −20° C., and then a CuCN.2LiCl solution (2.8 mL, 2.8 mmoles, 1 M in THF) is added. The reaction mixture is stirred for 10 min. Subsequently, a solution of (Z)-ethyl-3-iodobut-2-enoate (240 mg, 1.00 mmol) and Fe(acac)₃ (35 mg, 0.10 mmoles) dissolved in DME (3 mL) is added at once and the reaction mixture is then heated for 3 h to 80° C. The reaction is terminated by the addition of sat. NH₄Cl(aq.) and extracted with CH₂Cl₂ (3×40 mL). The combined organic phases are washed with sat. NH₄Cl(aq.)/NH₃ (9:1) (50 mL) and sat. NaCl(aq) (50 mL), dried over Na₂SO₄, filtered and the solvent is distilled-off under reduced pressure. Purification by column chromatography (pentane/diethylether=19:1) yielded (Z)-ethyl-3-(4-methoxyphenyl)but-2-enoate as a colourless oil (128 mg, 58%).

¹H-NMR (300 MHz, CDCl₃, 25° C.): δ=7.35 (d, J=8.6 Hz, 2H), 6.79 (d, J=8.6 Hz, 2H), 6.02 (q, J=1.2 Hz, 1H), 4.12 (q, J=7.0 Hz, 2H), 3.75 (s, 3H), 2.46 (d, J=1.3 Hz, 3H), 1.22 (t, J=7.2 Hz, 3H).

¹³C-NMR (75 MHz, CDCl₃, 25° C.): δ=167.01, 160.37, 154.79, 134.24, 127.57, 115.24, 113.75, 59.62, 55.21, 17.55, 14.28.

MS (70 eV, EI): m/z (%): 220 (95) [M], 191 (10), 175 (100), 148 (57), 132 (10), 115 (15), 91 (13), 77 (10).

HRMS for C₁₃H₁₆O₃(220,1099): found: 220.1088.

### REFERENCES AND NOTES

[1] (a) Metal catalyzed Cross-Coupling Reactions; F. Diederich; P. J. Stang Eds. Wiley-VCH, Weinheim, 1998; (b) J. Tsuji, Transition Metal Reagents and Catalysts. Innovations in Organic Synthesis; Wiley: Chichester, 1995; (c) Cross-Coupling Reactions. A Practical Guide; N. Miyaura Ed. Top. Curr. Chem. 2002, 219, Springer Verlag Berlin, Heidelberg, N.Y.
[2] Handbook of organopalladium chemistry for organic synthesis, Ed. E. Negishi, Wiley Interscience, New York, 2002.
[3] (a) C. Dai, G. C. Fu, J. Am. Chem. Soc. 2001, 123, 2719; (b) A. F. Littke, G. C. Fu, J. Am. Chem. Soc. 2001, 123, 6989; (c) A. F. Littke, L. Schwarz, G. C. Fu, J. Am. Chem. Soc. 2002, 124, 6343; (d) A. F. Littke, G. C. Fu, Angew. Chem. 2002, 114, 4350; Angew. Chem. Int. Ed. 2002, 41, 4176; (e) I. D. Hills, M. R. Netherton, G. C. Fu, Angew. Chem. 2003, 115, 5927; Angew. Chem. Int. Ed. 2003, 42, 5749; (g) A. C. Frisch, A. Zapf, O. Briel, B. Kayser, N. Shaikh, M. Beller, J. Mol. Catalysis. A 2004, 214, 231; (h) A. Tewari, M. Hein, A. Zapf, M. Beller, Synthesis 2004, 935; (i) F. Rataboul, A. Zapf, R. Jackstell, S. Harkal, T. Riermeier, A. Monsees, U. Dingerdissen, M. Beller, Chem. Eur. J. 2004, 10, 2983.
[4] (a) M. Kumada, Pure Appl. Chem. 1980, 52, 669; (b) T. -Y. Luh, Acc. Chem. Res. 1991, 24,257; (c) S. Sengupta, M. Leite, D. S. Raslan, C. Quesnelle, V. Snieckus, J. Org. Chem. 1992, 57, 4066; (d) A. F. Indolese, Tetrahedron Lett. 1997, 38, 3513; (e) E. Shirakawa, K. Yamasaki, T. Hiyama, Synthesis 1998,1544; (f) A. Sophia, E. Karlström, K. Itami, J. -E. Bäckvall, J. Am. Chem. Soc. 2000, 122, 6950; (h) J. Montgomery, Acc. Chem. Res. 2000, 33, 467; (i) R. Giovaninni, P. Knochel, J. Am. Chem. Soc. 1998, 120, 11186; (j) B. H. Lipshutz, Adv. Synth. Catal. 2001, 343, 313; (k) B. H. Lipshutz, G. Bulwo, F. Femandez-Lazaro, S. -K. Kim, R. Lowe, P. Mollard, K. L. Stevens, J. Am. Chem. Soc. 1999, 121, 11664.
[5] (a) M. Tamura, J. K. Kochi, J. Am. Chem. Soc. 1971, 93, 1487; (b) M. Tamura, J. K. Kochi, Synthesis 1971, 93, 303; (c) M. Tamura, J. K. Kochi, J. Organomet Chem. 1971, 31, 289; (d) M. Tamura, J. K. Kochi, Bull. Chem. Soc. Jpn. 1971, 44, 3063; (e) M. Tamura, J. K. Kochi, Synthesis 1971, 303; (f) J. K. Kochi, Acc. Chem. Res. 1974, 7, 351; (g) S. Neumann, J. K. Kochi, J. Org. Chem. 1975, 40, 599; (h) R. S. Smith, J. K. Kochi, J. Org. Chem. 1976, 41, 502; (i) Furthermore: G. Molander, B. Rahn, D.C. Shubert, S. E. Bonde, Tetrahedron Lett. 1983, 24, 5449.
[6] (a) G. Cahiez, S. Marquais, Pure Appl. Chem. 1996, 68, 669; (b) G. Cahiez, S. Marquais, Tetrahedron Lett. 1996, 37, 1773; (c) G. Cahiez, H. Avedissian, Synthesis 1998, 1199; (d) K. Shinokubo, K. Oshima, Eur. J. Org. Chem. 2004, 2081; (e) M. A. Fakhfakh, X. Franck, R. Hocquemiller, B. Figadère, J. Organomet. Chem. 2001, 624, 131; (f) M. Hocek, H. Dvoráková, J. Org. Chem. 2003, 68, 5773; (g) B. Holzer, R. W. Hoffmann, Chem. Comm. 2003, 732; (h) W. Dohle, F. Kopp, G. Cahiez, P. Knochel, Synlett 2001, 1901; (i) M. Hojo, Y. Murakami, H. Aihara, R. Sakuragi, Y. Baba, A. Hosomi, Angew. Chem. 2001, 113, 641; Angew. Chem. Int. Ed. 2001, 40, 621; (j) M. Nakamura, A. Hirai, E. Nakamura, J. Am. Chem. Soc. 2001, 122, 978; (k)E. Alvarez, T. Cuvigny, C. H. du Penhoat, M. Julia, Tetrahedron 1998, 44, 119; (l) V. Finandanese, G. Marchese, V. Martina, L. Ronzini, Tetrahedron Lett. 1984, 25, 4805.
[7] (a) A. Fürstner, A. Leitner, M. Méndez, H. Krause, J. Am. Chem. Soc. 2002, 124, 13856; (b) A. Fürstner, A. Leitner, Angew. Chem. 2002, 114, 632; Angew. Chem. Int. Ed. 2002, 41, 2003, 42, 308; (c) A. Fürstner, A. Leitner, Angew. Chem. 2003, 115, 320; Angew. Chem. Int. Ed. 2003, 42, 308; (d) G. Seidel, D. Laurich, A. Fürstner, J. Org. Chem. 2004, 69, 3950; (e) B. Scheiper, M. Bonnekessel, H. Krause, A. Fürstner, J. Org. Chem. 2004, 69, 3943; (f) A. Fürstner, D. De Souza, L. Parra-Rapado, J. T. Jensen, Angew. Chem. 2003, 115, 5516; Angew. Chem. Int Ed. 2003, 42, 5355; (g) M. Nakamura, K. Matsuo, S. Ito, E. Nakamura, J. Am. Chem. Soc. 2004, 126, 3686; (h) T. Nagano, T. Hayashi, Org. Lett. 2004, 6, 1297; (i) R. Martin, A. Fürstner, Angew. Chem. 2004, 116, 4045; Angew. Chem. Int. Ed. 2004, 43, 3955.
[8] K. Reddy, P. Knochel, Angew. Chem. 1996, 108, 1812; Angew. Chem. Int. Ed. 1996, 35, 1700.
[9 ] (a) B. H. Lipshutz, S. Sengupta, Org. React. 1992, 41, 135; (b) R. J. K. Taylor, Organocopper Reagents, Oxford University Press, Oxford, 1994; (c) N. Krause, Modern Organocopper Chemistry, Wiley VCH, Weinheim, 2002.
[10] (a) A. Krasovskiy, P. Knochel, Angew. Chem. 2004, 116, 3316; Angew. Chem. Int. Ed. 2004, 43, 3333; (b) For a review see: P. Knochel, W. Dohle, N. Gommermann, F. F. Kneisel, F. Kopp, T. Korn, I. Sapountzis, V. A. Vu, Angew. Chem. 2003, 115, 4438; Angew. Chem. Int. Ed. 2003, 42, 4302.
[11] P. Knochel, M. C. P. Yeh, S. C. Berk, J. Talbert, J. Org. Chem. 1988, 53, 2390.
[12] C. Piazza, P. Knochel, Angew. Chem. 2002, 114, 3263; Angew. Chem. Int. Ed. 2002, 41, 3263.

1. A process for the preparation of a compound of the general formula 4
R¹—Ar¹—Ar²—R² (4)
by reacting a compound of the general formula (1)
R¹—Ar¹—CuZMgY (1)
or a compound of the general formula (5)
R¹—Ar¹—CuZLi (5)
with a compound of the general formula (3)
R²—Ar²—X (3)
under the action of a Co or Fe catalyst in a solvent wherein
X may be a leaving group useful for nucleophilic substitution;
Y may be Cl, Br, I;
Z may be CN, Cl, Br, I, SCN, NR¹R², SR¹, PR¹R², alkyl, alkinyl;
R¹ and R² independently of each other may be one or more substituents selected from H; substituted or unsubstituted aryl or heteroaryl containing one or more heteroatoms; straight-chain, branched or cyclic substituted or unsubstituted alkyl, alkenyl, alkinyl; or derivatives thereof;
Ar¹ and Ar² independently of each other may be an aryl, condensed aryl, heteroaryl or condensed heteroaryl containing one or more heteroatoms; an alkenyl or alkinyl; or derivatives thereof.

2. The process according to claim 1 wherein the reaction is performed at a temperature between 0° C. and 150° C., preferably between 10° C. and 120° C., even more preferably between 20° C. and 100° C., most preferably between 25° C. and 80° C.

3. The process according to claim 1 wherein the catalyst comprises a Fe(III) complex, a Fe(III) salt, a Fe(II) complex, a Fe(II) salt, or a reduced form of a Fe salt or complex, preferably Fe(acac)₃.

4. The process according to claim 1 wherein the catalyst comprises a Co(II) or Co(III) catalyst or a reduced form of a Co salt or complex.

5. The process according to claim 4 wherein the catalyst is selected from the group comprising CoCl₂, CoBr₂, Co(OAc)₂, Co(Bzac)₂, CoBr₂dppe, Co(acac)₂ and Co(acac)₃, and preferably Co(acac)₂ is used.

6. The process according to any of the claims 4 or 5 wherein ethene and/or one or more ethene derivative(s), preferably electron-deficient ethene derivatives, particularly preferred maleic anhydride, tetracyano-ethylene, styrene or a styrene derivative, even more preferably an electron-deficient styrene derivative, and most preferably 4-fluorostyrene, is/are additionally added during the catalytic reaction.

7. A process according to claim 6 wherein the ethene or ethene derivative is added in an amount of 0-50 mole %, preferably 1-30 mole %, particularly preferred 5-25 mole %, most preferably 10-20 mole %, based on the molar amount of compound (3).

8. The process according to claim 4 or claim 5 wherein one or more salts, preferably tetrabutylammonium chloride, tetrabutylammonium bromide, potassium iodide, lithium iodide and/or most preferably tetrabutylammonium iodide are additionally added during the catalytic reaction.

9. A process according to claim 3 wherein X can preferably be F, Cl, Br, I, OTf, OTs, N₂⁺, more preferably Cl or Br, even more preferably I.

10. The process according to claim 4 wherein X can preferably be F, Cl, Br, I, OTf, OTs, N₂⁺, more preferably F, Cl, I or OTs, even more preferably Br.

11. The process according to claim 1 wherein a polar solvent or solvent mixture, preferably an etheric solvent or solvent mixture and most preferably a solvent or solvent mixture selected from the group comprising THF, DME, NMP, DMPU and DMAC is used as the solvent.

12. The process according to claim 1 wherein the compound (1) or (5) is added in a molar ratio of 0.9-5, preferably in a molar ratio of 1-3, even more preferably in a molar ratio of 1.2-2.5 based on the molar amount of compound (3).

13. The process according to claim 1 wherein Z preferably is CN.

14. The process according to claim 1 wherein R¹ and R² independently of each other can be a substituted or unsubstituted C₄-C₂₄ aryl or C₃-C₂₄ heteroaryl containing one or more heteroatoms such as B, O, N, S, Se, P; a straight-chain or branched, substituted or unsubstituted C₁-C₂₀ alkyl, C₁-C₂₀ alkenyl, C₁-C₂₀ alkinyl; or a substituted or unsubstituted C₃-C₂₀ cycloalkyl; or derivatives thereof.

15. The process according to claim 6 wherein one or more salts, preferably tetrabutylammonium chloride, tetrabutylammonium bromide, potassium iodide, lithium iodide and/or most preferably tetrabutylammonium iodide are additionally added during the catalytic reaction.
